(19) United States
(12) Patent Application Publication
(10) Pub. No.: US 2026/0166280 A1
(43) Pub. Date: Jun. 18, 2026

(54) SECUREMENT DEVICE FOR CATHETER, KIT, AND METHOD OF USE

(71) Applicant: Solventum Intellectual Properties Company, Maplewood, MN (US)

(72) Inventors: Ganesh P.D. Kannan, Bangalore (IN); Alan R. Dombrowski, Woodbury, MN (US); Krystal J. Scheibel, Minneapolis, MN (US); James M. Sieracki, Plymouth, MN (US); Timothy J. Nies, Stillwater, MN (US)

(73) Assignee: Solventum Intellectual Properties Company, Maplewood, MN (US)

(21) Appl. No.: 19/127,244

(22) PCT Filed: Oct. 30, 2023
(86) PCT No.: PCT/IB2023/060926

§ 371 Date: May 5, 2025

Related U.S. Application Data
(60) Provisional application No. 63/423,442, filed on Nov. 7, 2022.

(51) Int. Cl. A61M 25/02; A61B 17/70; A61M 25/00; A61M 25/01

(52) U.S. Cl. CPC A61M 25/02; A61B 17/7032; A61M 25/0067; A61M 25/0111; A61M 2025/024; A61M 2025/0253; A61M 2025/026; A61M 2025/0266; A61M 2025/0286

(57) ABSTRACT

A securement device for a cannula configured to be inserted into a skin of a patient at an insertion site is described. The securement device includes a base including a receiving member configured to be removably secured to the cannula. The base further includes a pair of arms extending from the receiving member and opposing each other. The securement device further includes a pair of anchor assemblies corresponding to the pair of arms and spaced apart from each other. Each of the pair of anchor assemblies is pivotally coupled to a corresponding arm from the pair of arms. Each of the pair of anchor assemblies is configured to be removably secured to the skin of the patient in a secured state. In the secured state of each of the pair of anchor assemblies, the base is pivotable relative to the pair of anchor assemblies while being secured to the cannula.

### TECHNICAL FIELD

The present disclosure relates to a securement device for a catheter, a kit including the securement device, and a method of using the securement device.

### BACKGROUND

Catheters are well known in medical applications and are used for various purposes, such as for administering medications and fluids to a user, obtaining blood tests, measuring venous pressure, etc. Generally, catheters are secured to a skin of the user to restrict movement thereof, e.g., to prevent the catheter from being pulled out, or otherwise moved in ways that may adversely impact a functioning of the catheter. Conventionally, catheters are secured to the skin of the user using catheter securement units, such as tapes, patches, and sutures. An improperly stabilized catheter may cause complications, such as dislodgement and accidental removal, phlebitis, occlusion/infiltration and leakage, bloodstream infections, etc.

Conventional catheter securement units, such as tapes, patches, etc., suffer from high rates of dislodgment. Further, use of such catheter securement units often allows partial or micro movement of the catheter. Use of sutures to secure the catheter in place may lead to suture related infection and complications. Additionally, the conventional catheter securement units may not allow a canulation site (insertion point) on the skin of the user to be accessed all around for maintenance purposes without dislodging the catheter or a hub of the catheter. The canulation site is only accessible after releasing the catheter from any current securement unit. This may lead to pistoning of the catheter, i.e., a mechanical motion of the catheter in and out of the skin of the user.

### SUMMARY

In a first aspect, the present disclosure provides a securement device for a catheter having a cannula configured to be inserted into a skin of a patient at an insertion site. The securement device includes a base including a receiving member configured to be removably secured to the cannula. The base further includes a pair of arms extending from the receiving member and opposing each other. The securement device further includes a pair of anchor assemblies corresponding to the pair of arms and spaced apart from each other. Each of the pair of anchor assemblies is pivotally coupled to a corresponding arm from the pair of arms of the base. Each of the pair of anchor assemblies is configured to be removably secured to the skin of the patient in a secured state. In the secured state of each of the pair of anchor assemblies, the base is pivotable relative to the pair of anchor assemblies while being secured to the cannula.

In a second aspect, the present disclosure provides a kit including a catheter. The catheter includes a cannula configured to be inserted into a skin of a patient at an insertion site. The kit further includes a securement device including a base. The base includes a receiving member configured to be removably secured to the cannula. The base further includes a pair of arms extending from the receiving member and opposing each other. The securement device further includes a pair of anchor assemblies corresponding to the pair of arms and spaced apart from each other. Each of the pair of anchor assemblies is pivotally coupled to a corresponding arm from the pair of arms of the base. Each of the pair of anchor assemblies is configured to be removably secured to the skin of the patient in a secured state. In the secured state of each of the pair of anchor assemblies, the base is pivotable relative to the pair of anchor assemblies while being secured to the cannula.

In a third aspect, the present disclosure provides a method of using a securement device with a catheter having a cannula inserted into a skin of a patient at an insertion site. The securement device includes a base having a receiving member, a pair arms extending from the receiving member, and a pair of anchor assemblies corresponding to the pair of arms. Each of the pair of anchor assemblies is pivotally coupled to a corresponding arm from the pair of arms of the base. The method includes removably securing the cannula of the catheter to the receiving member of the base. The method further includes removably securing each of the pair of anchor assemblies to the skin of the patient in a secured state. The method further includes pivoting the base away from the skin of the patient while the base is secured to the cannula.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 illustrates a schematic perspective view of a catheter, according to an embodiment of the present disclosure;

FIG. 2 illustrates a schematic perspective view of a securement device detached from the catheter, according to an embodiment of the present disclosure;

FIG. 3 illustrates a schematic exploded perspective view of the securement device, according to an embodiment of the present disclosure;

FIG. 4 illustrates a schematic perspective view of the securement device and the catheter where a cannula of the catheter is removably secured to a receiving member of a base of the securement device, according to an embodiment of the present disclosure;

FIG. 5 illustrates a schematic perspective view of the securement device and the catheter where a cap of the securement device is detachably coupled to the receiving member, according to an embodiment of the present disclosure;

FIG. 6A-6C illustrate partial schematic perspective views of the securement device in a released state, an intermediate state, and a secured state, respectively, of a pair of anchor assemblies, according to an embodiment of the present disclosure;

FIG. 7 illustrates a schematic perspective view of the securement device in the secured state of each of the pair of anchor assemblies, according to an embodiment of the present disclosure;

FIG. 8 illustrates a schematic perspective view of the securement device in the secured state of each of the pair of anchor assemblies and in a maintenance state of the base, according to an embodiment of the present disclosure;

FIG. 9 illustrates a schematic perspective view of the securement device where a longitudinal axis of the receiving member is orthogonal to a support plane defined by each of the pair of anchor assemblies, according to an embodiment of the present disclosure;

FIG. 10 illustrates a schematic block diagram of a kit, according to an embodiment of the present disclosure; and

FIG. 11 is a flowchart illustrating a method of using the securement device with the catheter, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As used herein, the term “coupled” generally means either a direct connection between two or more elements that are connected, or an indirect connection through one or more passive or active intermediary devices.

As used herein, the term “pivot” generally means to rotate or turn a component about a fixed axis.

As used herein, the term “pivotally coupled” generally refers to elements which are coupled in a way that permits one element to pivot with respect to another element.

As used herein, the term “removably secured” or “detachably coupled” generally means that a component may be secured in a fixed position to a second component and may be removed from the second component without causing structural damage to either components.

As used herein, the term “living hinge” generally refers to a flexible hinge that is made from the same material as the elements it is pivotally coupling together rather than another material or mechanism separate from the elements being connected.

As used herein, the term “securement” generally means the state of being secured, an act or instance of being secured, or a result of securing something.

As used herein, the term “slidably engage” generally refers to movement of one surface over a second surface while maintaining smooth continuous contact between the two surfaces.

As used herein, the term “catheter” is used throughout the specification to describe a thin tube extruded from medical grade materials serving a broad range of functions. Catheters are medical devices that may be inserted in a body of a user to treat diseases or perform a surgical procedure. By modifying the material or adjusting the way catheters are manufactured, it is possible to tailor catheters for cardiovascular, urological, gastrointestinal, neurovascular, and ophthalmic applications. Catheters may be inserted into a body cavity, a duct, or a vessel. Functionally, catheters may allow drainage, administration of fluids or gases, access for surgical instruments, and a wide variety of other tasks depending on the type of catheter. The process of inserting a catheter may be referred to as catheterization. In most cases, a catheter is a thin, flexible tube (“soft” catheter) though catheters are available in varying levels of stiffness depending on the application. A catheter left inside the body, either temporarily or permanently, may be referred to as an indwelling catheter. A permanently inserted catheter may be referred to as a permcath. A range of polymers may be used for manufacturing catheters, including, but not limited to, silicone rubber, nitinol, nylon, polyurethane, polyethylene terephthalate (PETE) latex, and/or thermoplastic elastomers.

As used herein, the term “maintenance” or “maintenance purpose” generally refers to a procedure that includes cleaning and/or sanitizing of a skin of a patient proximal to an insertion site. Maintenance may be carried out by a medical practitioner at regular intervals during, for example, an intravenous therapy.

As used herein, the term “pistoning” or “pistoning movement” refers to a movement of a cannula of a catheter relative to an insertion site.

As used herein, all numbers should be considered modified by the term “about”. As used herein, “a,” “an,” “the,” “at least one,” and “one or more” are used interchangeably.

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties).

As used herein, the term “configured to” and like is at least as restrictive as the term “adapted to” and requires actual design intention to perform the specified function rather than mere physical capability of performing such a function.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials comprises less than about 10 weight % of each of the first and second materials.

As used herein, “at least one of A and B” should be understood to mean “only A, only B, or both A and B”.

Unless specified or limited otherwise, the terms “attached,” “connected,” and variations thereof, are used broadly and encompass both direct and indirect attachments, connections, and couplings.

The present disclosure provides a securement device for a catheter having a cannula configured to be inserted into a skin of a patient at an insertion site. The securement device includes a base including a receiving member configured to be removably secured to the cannula. The base further includes a pair of arms extending from the receiving member and opposing each other. The securement device further includes a pair of anchor assemblies corresponding to the pair of arms and spaced apart from each other. Each of the pair of anchor assemblies is pivotally coupled to a corresponding arm from the pair of arms of the base. Each of the pair of anchor assemblies is configured to be removably secured to the skin of the patient in a secured state. In the secured state of each of the pair of anchor assemblies, the base is pivotable relative to the pair of anchor assemblies while being secured to the cannula.

The securement device of the present disclosure may secure and stabilize the catheter firmly onto the skin of the patient. Further, the securement device may restrict a movement of the cannula with respect to the skin of the patient during normal usage. Specifically, the pair of anchor assemblies may prevent dislodgement of the catheter or the cannula when removably secured to the skin of the patient. Moreover, the pair of anchor assemblies may allow ease of removal of the securement device from the the skin of the patient.

In the secured state of each of the pair of anchor assemblies, the securement device may allow the base to be pivoted relative to the pair of anchor assemblies while being secured to the cannula, thereby allowing all around access to the insertion site for cleaning and maintenance purposes without the need to dislodge the catheter from the securement device. Further, the securement device may be secured to the skin of the patient away from the insertion site allowing minimal interference with the insertion site. The securement device may be used for extended periods of time without causing discomfort to the patient, thereby improving a user experience.

Additionally, the securement device may allow a length of the cannula between the base and the insertion site to be fixed, thereby eliminating pistoning of the cannula during pivoting movement of the base relative to the pair of anchor assemblies. Further, the securement device of the present disclosure may eliminate the need for tapes, patches, and/or sutures to secure the catheter to the skin of the patient, thereby preventing medical adhesive-related skin injury (MARSI) complications. Further, the base of the securement device may removably secure cannulas of various sizes and geometries, thereby enabling compatibility with multiple brands and applications.

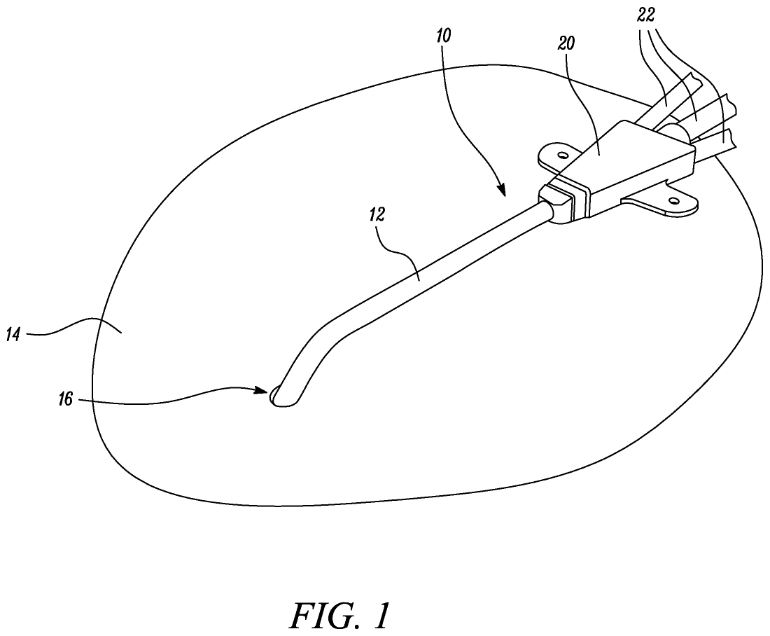

Referring now to Figures, FIG. 1 illustrates a schematic perspective view of a catheter 10. The catheter 10 includes a cannula 12 configured to be inserted into a skin 14 of a patient (not shown) at an insertion site 16. As shown in FIG. 1, the catheter 10 is at least partially disposed on the skin 14 of the patient. In some cases, the patient utilizing the catheter 10 may be undergoing an intravenous therapy.

In the following description, “inserted into the skin” includes insertion through the skin. A distal end of the catheter 10 or the cannula 12 may be located at any medically desired anatomical site, including, as non-limiting examples, lumens of veins and arteries, atria and ventricles of the heart, a ventral body cavity, a spinal canal, a subarachnoid space, an intracranial space, and a fat or muscle tissue.

In some examples, the catheter 10 includes a catheter hub 20 and at least one lumen 22 at least partially received within the catheter hub 20. Further, the cannula 12 is at least partially received within the catheter hub 20. In the illustrated embodiment of FIG. 1, the at least one lumen 22 includes three lumens 22. In other words, the catheter 10 is a triple lumen catheter. However, the number of the lumens 22 may vary based on desired application and/or infusion attributes. The at least one lumen 22 is fluidly connected to the cannula 12 through the catheter hub 20.

In some examples, the catheter 10 may include a central venous catheter (CVC). The CVC may be referred to as a central line, a central venous line, or a central venous access catheter. The CVC may be used to access large, centrally located veins (not shown), which is often required for critically ill patients, for patients requiring prolonged intravenous therapies for reliable vascular access, and to administer fluids that may harm smaller peripheral veins. Therefore, in some examples, at least a portion of the cannula 12 may be inserted into veins located at a neck (e.g., an internal jugular vein), into veins located at a chest (e.g., a subclavian vein or axillary vein), or into veins located at a groin (e.g., a femoral vein) of the patient. In some examples, the catheter 10 may include a peripherally inserted central catheter (PICC). The PICC may be suitable for insertion into veins located at an arm of the patient. However, the catheter 10 may include any suitable type of catheter, which may be selected based on desired application attributes.

In some examples, the catheter 10 may be used to administer an intravenous therapy (e.g., administration of medication or fluids for parenteral nutrition), to obtain blood for analysis, and/or to provide an access point for blood-based treatments, such as dialysis or apheresis. The catheter 10 may further be used to measure one or more properties of the blood (e.g., a “central venous oxygen saturation”), administer fluid or blood products for a large volume resuscitation, and/or measure a central venous pressure.

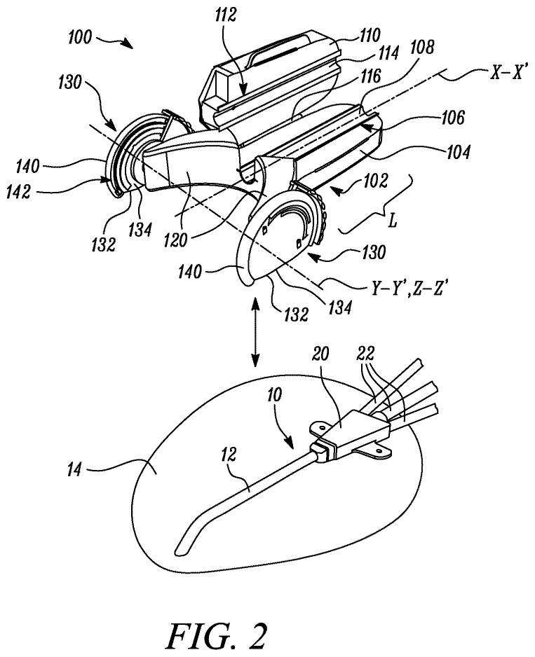

FIG. 2 illustrates a schematic perspective view of a securement device 100 for the catheter 10. The securement device 100 is detached and spaced apart from the catheter 10 in FIG. 2. The securement device 100 includes a base 102. The base 102 includes a receiving member 104 configured to be removably secured to the cannula 12. In some examples, the receiving member 104 of the base 102 is configured to be removably secured to the cannula 12 after the cannula 12 is inserted into the skin 14 (or a central vein) of the patient at the insertion site 16.

In some examples, the securement device 100 further includes a cap 110 configured to be detachably coupled to the receiving member 104. In the illustrated embodiment of FIG. 2, the cap 110 is shown uncoupled from the receiving member 104. In some examples, the base 102 further includes a living hinge 116 pivotally coupling the cap 110 to the receiving member 104. In some examples, the cap 110 may pivot relative to the receiving member 104 for receiving the cannula 12 between the cap 110 and the receiving member 104. In some examples, the cap 110 and the receiving member 104 are configured to receive the cannula 12 therebetween after insertion of the cannula 12 into the skin 14 of the patient.

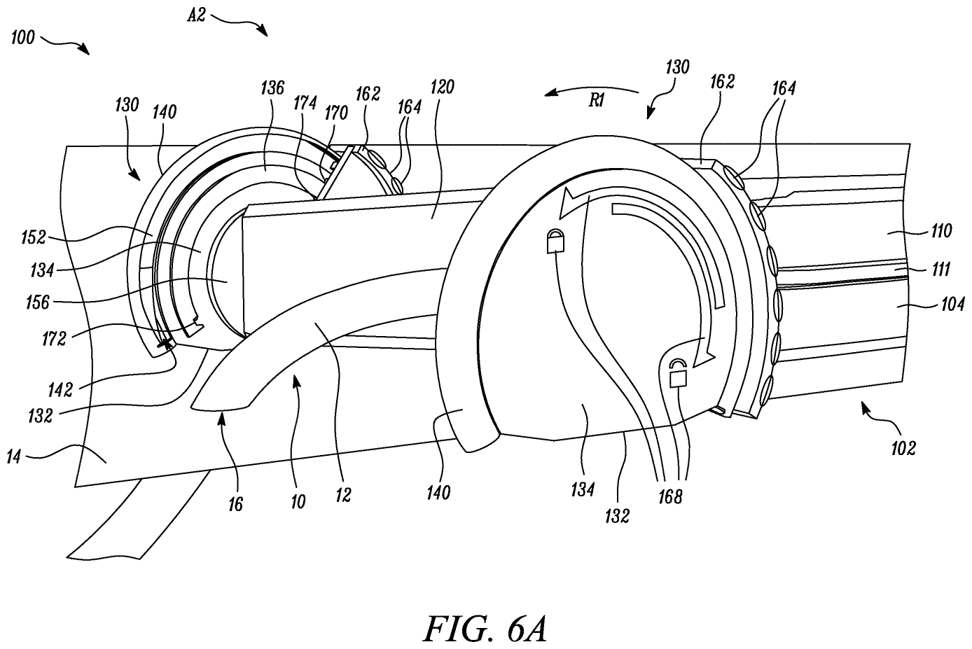

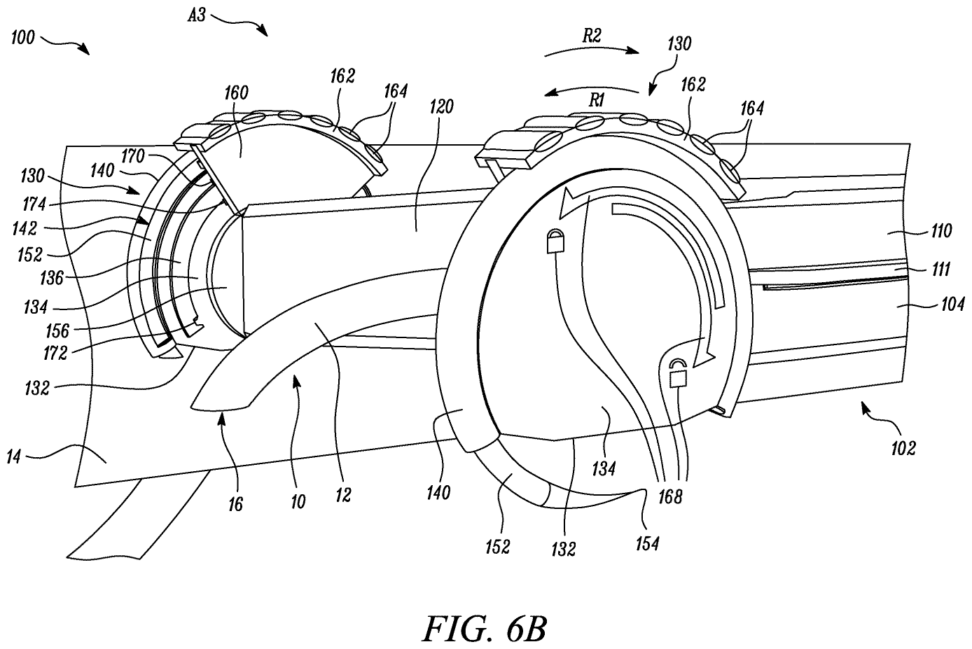

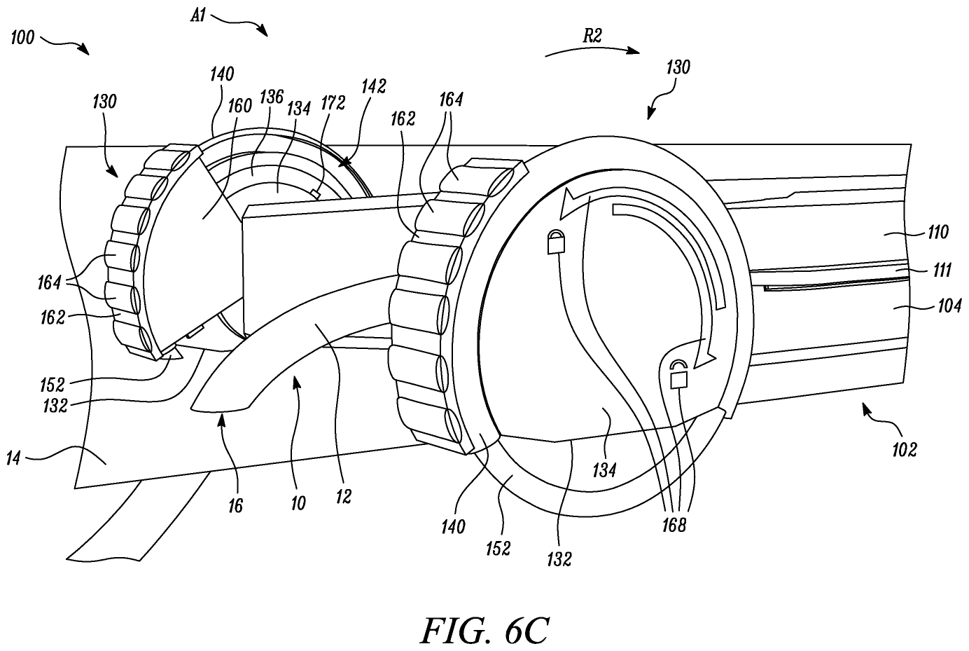

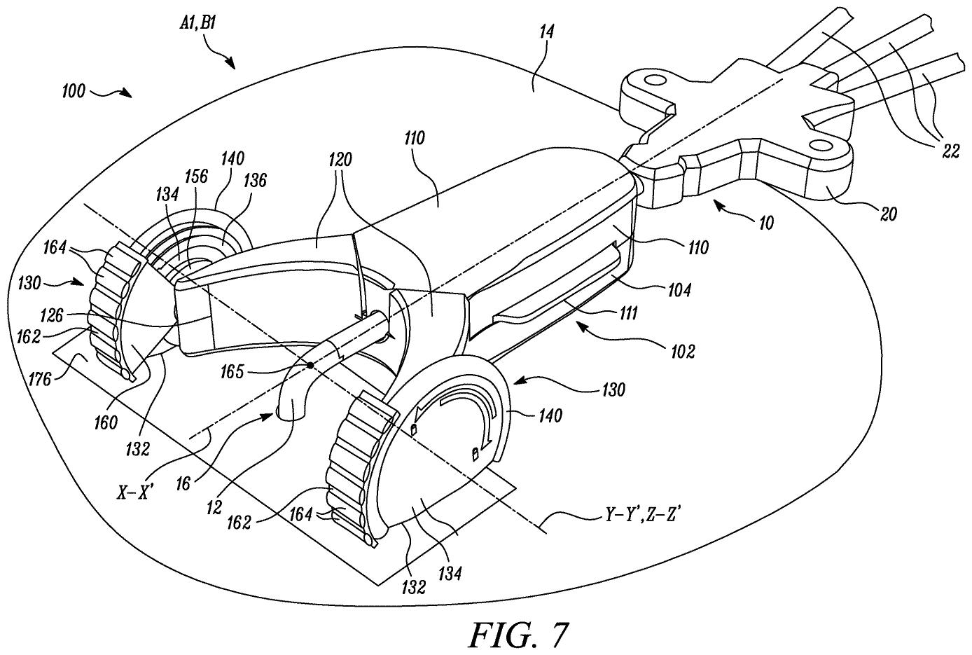

The base 102 further includes a pair of arms 120 extending from the receiving member 104 and opposing each other. However, it should be understood that the base 102 may include any number of the arms 120. In some examples, each of the pair of arms 120 may be arcuate. The securement device 100 further includes a pair of anchor assemblies 130 corresponding to the pair of arms 120 and spaced apart from each other. Each of the pair of anchor assemblies 130 is pivotally coupled to a corresponding arm 120 from the pair of arms 120 of the base 102. Each of the pair of anchor assemblies 130 is configured to be removably secured to the skin 14 of the patient in a secured state A1 (shown in FIGS. 6C and 7). In some examples, each of the pair of anchor assemblies 130 is configured to be removably secured to the skin 14 of the patient after removably securing the cannula 12 to the receiving member 104.

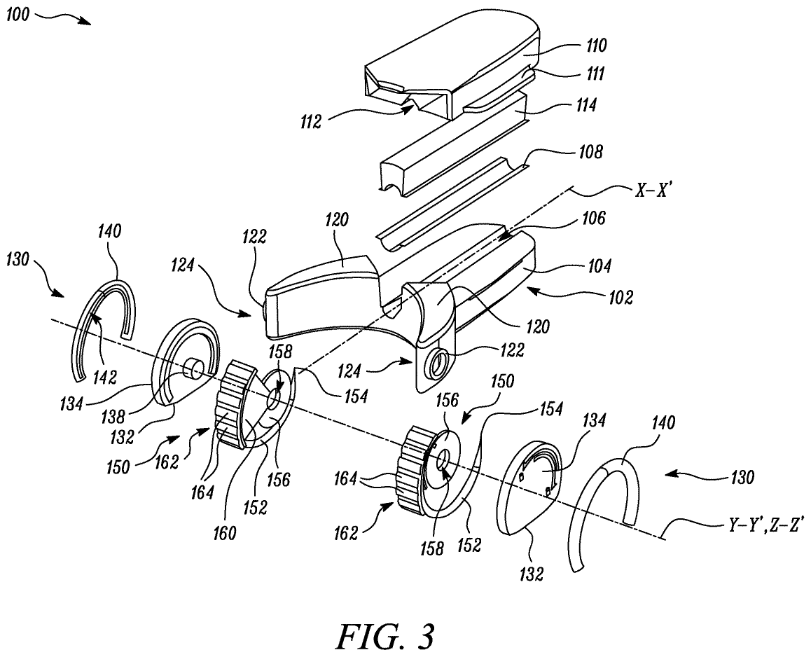

FIG. 3 illustrates a schematic exploded perspective view of the securement device 100. Referring now to FIGS. 2 and 3, in some examples, the receiving member 104 includes a first slot 106 configured to at least partially and removably receive the cannula 12 therein. In some examples, the securement device 100 further includes a first sleeve 108 at least partially received within the first slot 106. In some examples, the cap 110 includes a second slot 112. In some examples, a second sleeve 114 is at least partially received within the second slot 112. It should be understood that the dimensions and geometry of the first sleeve 108 and the second sleeve 114 may vary based on application requirements.

In some examples, the receiving member 104 further includes a longitudinal axis X-X′ extending along its length L (shown in FIG. 2). In some examples, the first slot 106 of the receiving member 104 at least partially extends along the longitudinal axis X-X′. Further, the second slot 112 of the cap 110 also at least partially extends along the longitudinal axis X-X′. In some examples, the longitudinal axis X-X′ corresponds to an axis of symmetry of the base 102, such that the pair of arms 120 are symmetrically arranged about the longitudinal axis X-X′. In some examples, each of the pair of arms 120 curves away from the longitudinal axis X-X′. Moreover, the longitudinal axis X-X′ may also align with the insertion site 16 on the skin 14 of the patient. In some examples, each of the pair of arms 120 is pivotable about a common pivot axis Y-Y′ that is substantially orthogonal to the longitudinal axis X-X′.

In some examples, each of the pair of anchor assemblies 130 includes a planar support surface 132 configured to be disposed on the skin 14 of the patient. In some examples, the planar support surface 132 may allow each of the pair of anchor assemblies 130 to be seated on the skin 14 of the patient. In some examples, each of the pair of anchor assemblies 130 further includes an anchor wheel 134 pivotally coupled to the corresponding arm 120. Specifically, the anchor wheel 134 includes the planar support surface 132. For example, a portion of the anchor wheel 134 may be removed to form the planar support surface 132. Alternatively, the anchor wheel 134 may be a molded part including the planar support surface 132.

In some examples, the anchor wheel 134 of each of the pair of anchor assemblies 130 includes a protrusion 138 and each arm 120 includes a collar 122 proximal to an end 124 distal to the receiving member 104. The protrusion 138 corresponding to only one of the pair of anchor assemblies 130 and the collar 122 corresponding to only one of the pair of arms 120 are visible in FIG. 3. In some examples, the collar 122 of each arm 120 at least partially and rotatably receives the protrusion 138 of the anchor wheel 134 of the corresponding anchor assembly 130 to pivotally couple each arm 120 to the corresponding anchor assembly 130. In some examples, the collar 122 of each arm 120 may retain the protrusion 138 of the anchor wheel 134 of the corresponding anchor assembly 130 to pivotally couple the arm 120 to the corresponding anchor assembly 130. In some examples, each arm 120 is pivotable relative to the corresponding anchor assembly 130 along the common pivot axis Y-Y′. In some examples, the common pivot axis Y-Y′ may pass through the protrusion 138 of the anchor wheel 134 of each of the pair of anchor assemblies 130.

In some examples, each of the pair of anchor assemblies 130 further includes an anchor guide 140 fixedly coupled to and disposed around the anchor wheel 134. In some examples, the anchor guide 140 may be disposed around a circumference of the anchor wheel 134 adjacent the planar support surface 132. In some examples, the anchor guide 140 may be molded to the anchor wheel 134, for instances where the anchor guide 140 and the anchor wheel 134 are made of polymeric material, or formed on the anchor wheel 134, for instances where the anchor guide 140 and the anchor wheel 134 are made of metallic material. In some examples, the anchor guide 140 may be in the form of an arcuate hollow tube. In some examples, the anchor guide 140 includes an arcuate slot 142 extending therethrough.

In some examples, each of the pair of anchor assemblies 130 further includes an anchor ring 150 including an arcuate penetrating portion 152 slidably and at least partially received within the arcuate slot 142 of the anchor guide 140, such that the anchor ring 150 is rotatable relative to the anchor guide 140 along the arcuate slot 142. In some examples, the anchor ring 150 of each of the pair of anchor assemblies 130 is rotatable about a common rotation axis Z-Z′. Further, in some examples, the common pivot axis Y-Y′ is coaxial with the common rotation axis Z-Z′. In some examples, the anchor ring 150 may be made of plastic (e.g., a molded part) or made of a medical grade stainless steel.

In some examples, the anchor ring 150 further includes a coupling portion 156 defining an aperture 158 therethrough and disposed adjacent to the anchor wheel 134. In some examples, the aperture 158 of the coupling portion 156 at least partially receives the protrusion 138 of the anchor wheel 134 therethrough to rotatably couple the anchor ring 150 to the anchor wheel 134. Thus, the anchor ring 150 is rotatable relative to the anchor wheel 134 as well as the anchor guide 140 about the common rotation axis Z-Z′. In some examples, the coupling portion 156 is disc-shaped.

In some examples, the anchor ring 150 further includes an intermediate portion 160 extending radially from the coupling portion 156. In some examples, the arcuate penetrating portion 152 extends from the intermediate portion 160 radially distal to the coupling portion 156. In some examples, the arcuate penetrating portion 152 further at least partially extends from the intermediate portion 160 about the common rotation axis Z-Z′. In some examples, the arcuate penetrating portion 152 includes a sharp tip 154 configured to removably penetrate the skin 14 of the patient. In some examples, the sharp tip 154 is disposed at an end of the arcuate penetrating portion 152 away from the intermediate portion 160.

In some examples, the anchor wheel 134 may act as a guide for the anchor ring 150 to rotate about the common rotation axis Z-Z′ without slipping out of the corresponding anchor assembly 130. In some examples, the anchor ring 150 further includes a grip pad 162 coupled to and at least partially disposed around the intermediate portion 160. In some examples, the grip pad 162 includes a plurality of ridges 164 configured to be manually gripped for rotating the anchor ring 150 relative to the anchor guide 140. In some examples, the intermediate portion 160 tapers in a radially inward direction from the grip pad 162 to the coupling portion 156.

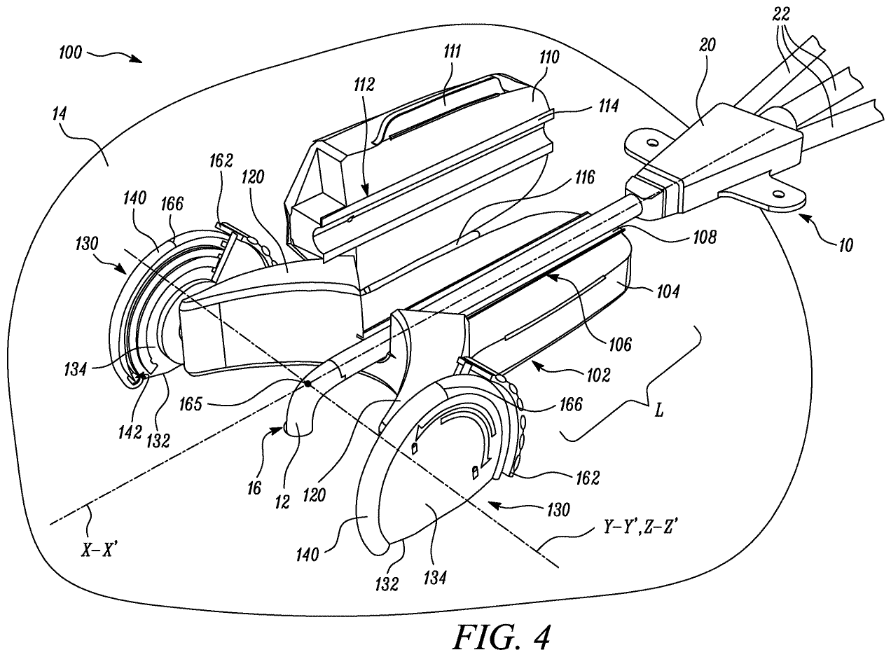

FIG. 4 illustrates a schematic perspective view of the securement device 100 and the catheter 10 where the cannula 12 is removably secured to the receiving member 104. Further, as shown in FIG. 4, the cap 110 of the securement device 100 is uncoupled from the receiving member 104. In some examples, the securement device 100 may be appropriately positioned under the catheter 10 after the cannula 12 is inserted into the skin 14 of the patient at the insertion site 16 before removably securing the cannula 12 to the receiving member 104. In some examples, the planar support surface 132 of each of the pair of anchor assemblies 130 engages with the skin 14 of the patient as the securement device 100 is positioned on the skin 14.

In some examples, the securement device 100 may be positioned on the skin 14 of the patient such that the securement device 100 is aligned with the insertion site 16 and the cannula 12 is at least partially disposed between the pair of arms 120. Further, the securement device 100 may be positioned such that common pivot axis Y-Y′ or the common rotation axis Z-Z′ is aligned with the insertion site 14, and the longitudinal axis X-X′ intersects with the common pivot axis Y-Y′ directly above the insertion site 16. Specifically, the longitudinal X-X′ and the common pivot axis Y-Y′ intersects each other at an intersection point 165 located directly above the insertion site 16.

In some examples, the anchor guide 140 of each of the pair of anchor assemblies 130 includes an alignment mark 166 aligned with the insertion site 16. The alignment mark 166 may allow a clinician to appropriately locate the securement device 100 on the skin 14 of the patient relative to the insertion site 16. In some examples, the alignment mark 166 may also be aligned with the common pivot axis Y-Y′ or the common rotation axis Z-Z′. In some examples, the clinician may appropriately adjust a length of the cannula 12 disposed between the receiving member 104 and the insertion site 16 before removably securing the cannula 12 to the receiving member 104. Further, the cannula 12 is at least partially and removably received in the first slot 106 of the receiving member 104.

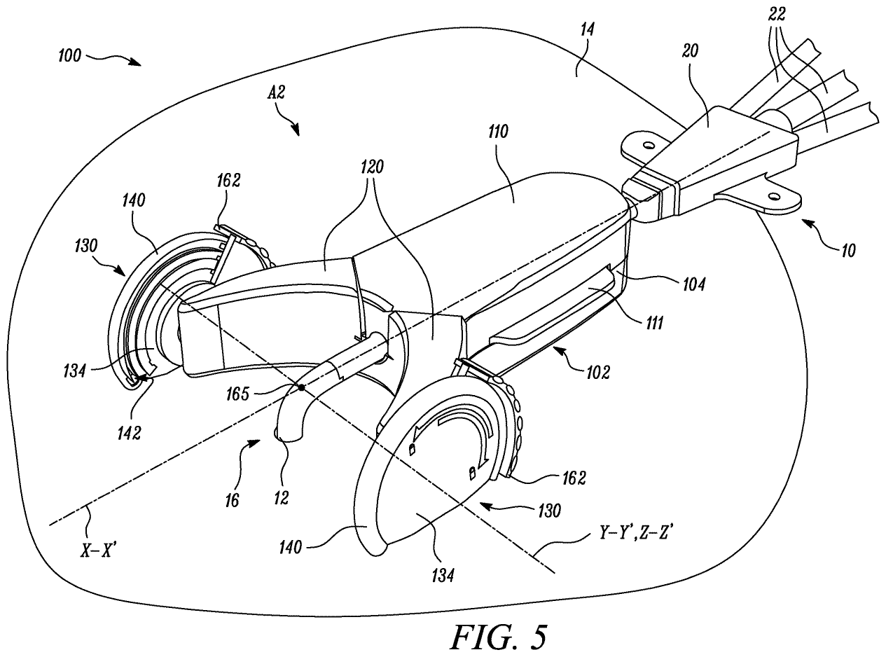

FIG. 5 illustrates a schematic perspective view of the securement device 100 and the catheter 10 where the cap 110 is detachably coupled to the receiving member 104. Once the securement device 100 is positioned on the skin 14 of the patient, the clinician may rotate the cap 110 and detachably secure the cap 110 to the receiving member 104. In some examples, the cap 110 is pivotable relative to receiving member 104 through the living hinge 116 (shown in FIG. 4) from an uncoupled state (shown in FIG. 4) to a coupled state (shown in FIG. 5) for detachably coupling the cap 110 to the receiving member 104.

In some examples, the cap 110 is detachably coupled to the receiving member 104 by a snap-fit connection. However, it should be understood that the cap 110 may be detachably coupled to the receiving member 104 via any other suitable coupling mechanism. In some examples, the cap 110 includes a lip 111 configured to be manually gripped for releasing the cap 110 from the receiving member 104. In some examples, the lip 111 may extend away from the cap 110 allowing the lip 111 to be manually gripped for applying a force to release the cap 110 from the receiving member 104. In some examples, the cap 110 may be released from the receiving member 104, e.g., when the securement device 100 is required to be discarded after use.

Referring to FIGS. 4 and 5, in some examples, upon detachable coupling of the cap 110 to the receiving member 104, the second slot 112 is at least partially aligned with the first slot 106. In some examples, upon insertion of the cannula 12 in the first slot 106 and detachable coupling of the cap 110 to the receiving member 104, the first and second slots 106, 112 collectively and at least partially receive the cannula 12 therebetween, such that the cannula 12 is removably secured to the receiving member 104.

Further, upon insertion of the cannula 12 in the first slot 106 and detachable coupling of the cap 110 to the receiving member 104, each of the first sleeve 108 and the second sleeve 114 frictionally engages with the cannula 12, such that the cannula 12 is removably secured to the receiving member 104. In some examples, the first sleeve 108 and the second sleeve 114 may be soft high friction material sleeves that may snugly fit on to the cannula 12 when the cap 110 is detachably coupled to the receiving member 104. Further, each of the first sleeve 108 and the second sleeve 114 may resist any pull forces to keep the cannula 12 in place, thereby allowing a secure and stable fit for the cannula 12 between the cap 110 and the receiving member 104.

In some examples, the first sleeve 108 and the second sleeve 114 may be replaceable, thereby allowing the cannula 12 of various sizes and geometries to be removably secured to the receiving member 104. This may enable compatibility of the securement device 100 with multiple brands and applications. In some examples, upon insertion of the cannula 12 in the first slot 106 and detachable coupling of the cap 110 to the receiving member 104, the cannula 12 at least partially extends from the receiving member 104 along the longitudinal axis X-X′.

In some examples, the anchor ring 150 is rotatable relative to the corresponding arm 120 between the secured state A1 (shown in FIGS. 6C and 7) and a released state A2 (shown in FIGS. 5 and 6A) of the corresponding anchor assembly 130. In some examples, the anchor wheel 134 and the anchor guide 140 are configured to remain fixed on the skin 14 of the patient while the anchor ring 150 is configured to rotate.

FIGS. 6A and 6C illustrate partial schematic perspective views of the securement device 100 in the released state A2 and the secured state A1, respectively, of each of the pair of anchor assemblies 130, respectively. Further, FIG. 6B illustrates a partial schematic perspective view of the securement device 100 in an intermediate state A3 of each of the pair of anchor assemblies 130. Further, FIG. 7 illustrates a schematic perspective view of the securement device 100 in the secured state A1 of each of the pair of anchor assemblies 130.

Referring now to FIGS. 5-7, in the secured state A1 (shown in FIGS. 6C and 7) of each of the pair of anchor assemblies 130, the arcuate penetrating portion 152 at least partially penetrates the skin 14 of the patient in order to removably secure the corresponding anchor assembly 130 to the skin 14 of the patient. In some examples, the anchor ring 150 rotates relative to the corresponding arm 120 as each of the pair of anchor assemblies 130 moves from the released state A2 (shown in FIGS. 5 and 6A) to the secured state A1 (shown in FIGS. 6C and 7), thereby causing the arcuate penetrating portion 152 of the anchor ring 150 of each of the pair of anchor assemblies 130 to at least partially penetrate the skin 14 of the patient.

As shown in FIGS. 6B and 6C, in some examples, the arcuate penetrating portion 152 of the anchor ring 150 penetrates the skin 14 of the patient through the sharp tip 154. In some examples, the rotation of the anchor ring 150 relative to the anchor guide 140 may cause the sharp tip 154 of the arcuate penetrating portion 152 to pierce the skin 14 at one end of the anchor guide 140 and is received back into the anchor guide 140 at the other end, thereby forming a loop and securing each of the pair of anchor assemblies 130 to the skin 14 of the patient. Thus, each of the pair of anchor assemblies 130 may anchor itself onto the skin 14 of the patient with the help of the anchor rings 150. Further, the loop may form a hooking securement to hold the securement device 100 in place on the skin 14. In some examples, the penetrating points are disposed away from the insertion site 16, thereby ensuring no interference at the insertion site 16.

In some examples, the anchor ring 150 is rotatable along a first rotational direction R1 to switch the corresponding anchor assembly 130 from the released state A2 (shown in FIGS. 5 and 6A) to the secured state A1 (shown in FIGS. 6C and 7). In some examples, the clinician may apply a force on the plurality of ridges 164 of the grip pad 162 of the anchor ring 150 along the first rotational direction R1 to rotate the anchor ring 150 along the first rotational direction R1, thereby at least partially penetrating the arcuate penetrating portion 152 into the skin 14 of the patient. In some examples, the clinician may need to apply a force on the base 102 to keep it in place while rotating the anchor ring 150. For example, one hand may be used to press on the base 102 and the other hand may be used to rotate the anchor ring 150.

In some examples, the anchor ring 150 is rotatable along a second rotational direction R2 opposite to the first rotational direction R1 to switch the corresponding anchor assembly 130 from the secured state A1 (shown in FIGS. 6C and 7) to the released state A2 (shown in FIGS. 5 and 6A). In some examples, the clinician may apply a force on the plurality of ridges 164 of the grip pad 162 of the anchor ring 150 along the second rotational direction R2 to rotate the anchor ring 150 along the second rotational direction R2, thereby causing the arcuate penetrating portion 152 to rotate along the second rotational direction R2 and release from the skin 14 of the patient. In some examples, in the released state A2 (shown in FIGS. 5 and 6A) of each of the pair of anchor assemblies 130, the arcuate penetrating portion 152 is removed from the skin 14 of the patient, such that the corresponding anchor assembly 130 is released from the skin 14 of the patient.

In some examples, the anchor wheel 134 further includes an arcuate shoulder 136. In some examples, the anchor ring 150 includes a projection 170 (shown in FIGS. 6A and 6B) slidably engaging with the arcuate shoulder 136. In some examples, upon rotation of the anchor ring 150, the projection 170 of the anchor ring 150 slides along the arcuate shoulder 136, thereby guiding the rotation of the anchor ring 150 relative to the anchor wheel 134.

In some examples, the anchor wheel 134 further includes one or more first locking features 172. In the illustrated examples of FIGS. 6A-6C, the one or more first locking features 172 are in the form of a notch disposed on either ends of the arcuate shoulder 136. In some examples, the anchor ring 150 includes one or more second locking features 174 (shown in FIGS. 6A and 6B) complimentary to the one or more first locking features 172. In some examples, the one or more second locking features 174 are in the form of a protuberance configured to lockingly and releasably engage with the one or more first locking features 172.

In some examples, the one or more first locking features 172 are provided on the anchor wheel 134 of each of the pair of anchor assemblies 130 corresponding to each of the secured state A1 (shown in FIGS. 6C and 7) and the released state A2 (shown in FIGS. 5 and 6A). Thus, the one or more first and second locking features 172, 174 may allow the anchor ring 150 to be releasably locked in the secured state A1 and the released state A2, thereby preventing the anchor ring 150 from advancing or retreating further than required. Further, the one or more first and second locking features 172, 174 may prevent any unintentional movement of the anchor ring 150 relative to the anchor wheel 134 in the secured state A1. It should be understood that the one or more first and second locking features 172, 174 may include any other suitable locking mechanism based on application requirements.

In some examples, at least one of the anchor wheel 134 and the anchor guide 140 includes one or more indicia 168 indicative of the first rotational direction R1, the second rotational direction R2, the secured state A1 (shown in FIGS. 6C and 7), and the released state A2 (shown in FIGS. 5 and 6A). In the illustrated examples of FIGS. 5-7, the anchor wheel 134 includes the one or more indicia 168 indicative of the first rotational direction R1, the second rotational direction R2, the secured state A1, and the released state A2.

Referring now to FIG. 7, in the secured state A1 of each of the pair of anchor assemblies 130, the base 102 is pivotable relative to the pair of anchor assemblies 130 while being secured to the cannula 12. In some examples, each of the pair of anchor assemblies 130 defines a support plane 176 parallel to the common pivot axis Y-Y′. In some examples, in a home state B1 of the base 102, the longitudinal axis X-X′ is parallel to the support plane 176 while the base 102 is secured to the cannula 12. In some examples, each of the pair of arms 120 includes an alignment line 126 orthogonal to the longitudinal axis X-X′. In some examples, in the home state B1 of the base 102, the alignment line 126 is further orthogonal to the common pivot axis Y-Y′. Therefore, in the home state B1, the base 102 is disposed on the skin 14 of the patient and the catheter 10 is immobilized. In some examples, the clinician may apply an additional window dressing (not shown) over the whole insertion site 16 to provide additional protection from infection.

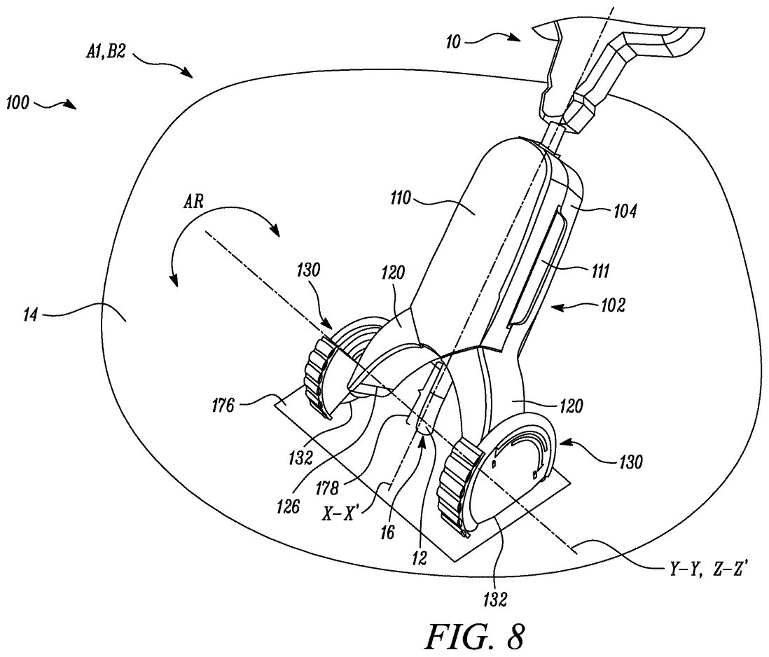

FIG. 8 illustrates a schematic perspective view of the securement device 100 in the secured state A1 of each of the pair of anchor assemblies 130 and in a maintenance state B2 of the base 102. In some examples, in the maintenance state B2 of the base 102, the longitudinal axis X-X′ is inclined to the support plane 176 while the base 102 is secured to the cannula 12. In some examples, the base 102 is pivotable relative to the pair of anchor assemblies 130, thereby moving the base 102 from the home state B1 (shown in FIG. 7) to the maintenance state B2. The alignment line 126 may be obliquely inclined to the longitudinal axis X-X′ based on an oblique angle of rotation of the base 102 from the home state B1.

In some examples, each of the pair of arms 120 is rotatable in an angular range AR of about 180 degrees relative to the corresponding anchor assembly 130. In other words, each of the pair of arms 120 is rotatable in the angular range AR of about 180 degrees relative to the skin 14 of the patient. In some examples, the planar support surface 132 of each of the pair of anchor assemblies 130 may remain engaged with the skin 14 of the patient while the base 102 is pivoted relative to the pair of anchor assemblies 130.

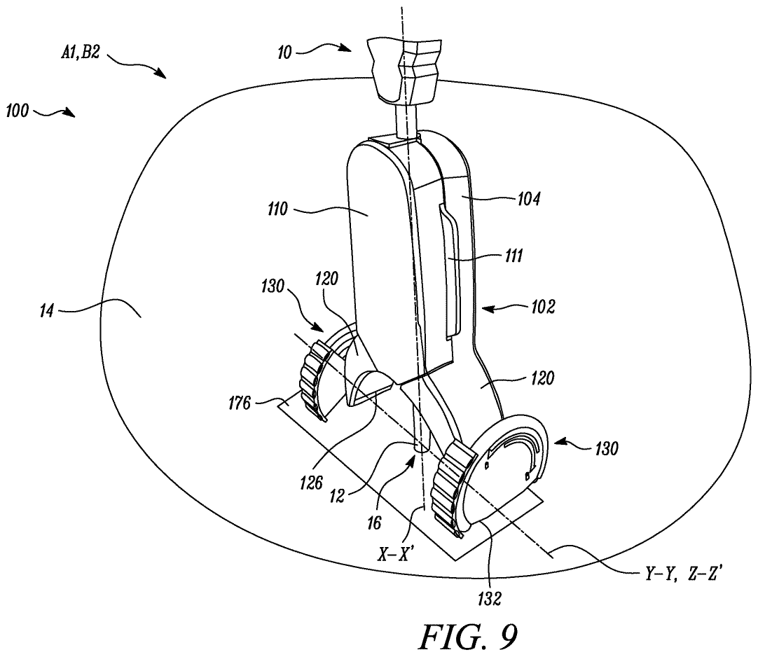

FIG. 9 illustrates a schematic perspective view of the securement device 100 where the longitudinal axis X-X′ of the receiving member 104 is orthogonal to the support plane 176 defined by each of the pair of anchor assemblies 130. In the illustrated example of FIG. 9, the securement device 100 may allow all around access to the insertion site 16 for cleaning or maintenance purposes. In some examples, the securement device 100 may allow access to the insertion site 16 without removing the securement device 100 from the skin 14 of the patient or the cannula 12 of the catheter 10 from the securement device 100. In some examples, the base 102 may be pivoted back to the home state B1 (shown in FIG. 7) after the required cleaning or maintenance.

Referring now to FIGS. 8 and 9, in some examples, an exposed length 178 (shown in FIG. 8) of the cannula 12 from the receiving member 104 to the insertion site 16 is substantially constant during pivoting of the base 102 relative to the pair of anchor assemblies 130. In some examples, the exposed length 178 may remain substantially constant since the insertion site 16 is disposed at a focal point of the arc formed by each of the pair of arms 120. Thus, the securement device 100 may prevent pistoning of the catheter 10 during the pivoting movement of the base 102 relative to the pair of anchor assemblies 130.

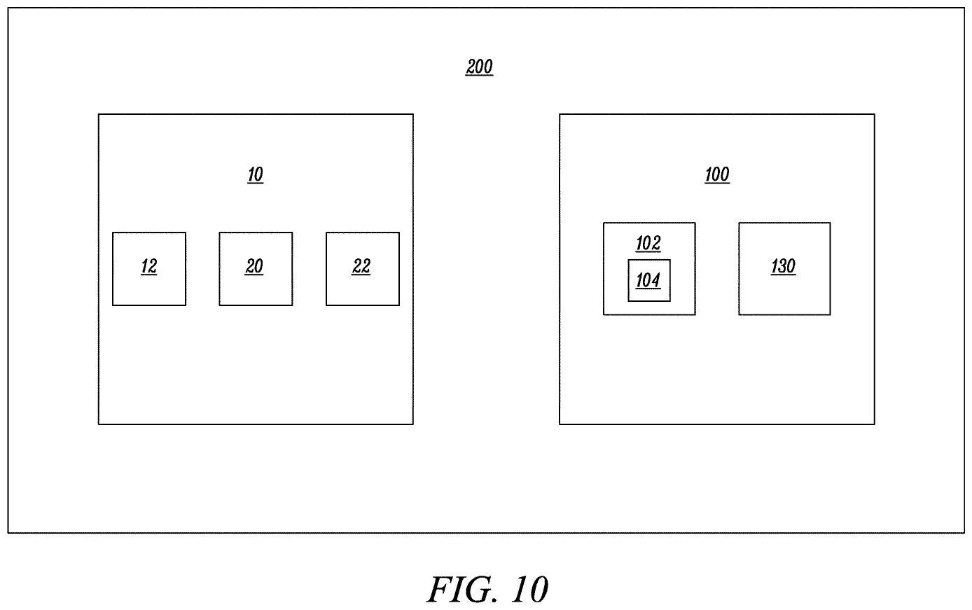

FIG. 10 illustrates a schematic block diagram of a kit 200. Referring now to FIGS. 1-10, the kit 200 includes the catheter 10 including the cannula 12 configured to be inserted into the skin 14 of the patient at the insertion site 16. The kit 200 further includes the securement device 100. The securement device 100 is used for securing the catheter 10 to the skin 14 of the patient. In some examples, the cannula 12 of the catheter 10 is configured to be removably secured to the receiving member 104 of the base 102. The catheter 10 may therefore be secured and stabilized on the skin 14 of the patient.

The securement device 100 includes the base 102 and the pair of anchor assemblies 130. In some embodiments, the kit 200 may further include gloves (not shown), a sterilizing material (not shown), and a cloth or other absorbent material (not shown). In some embodiments, the kit 200 may further include cleaning articles (not shown), such as cleaning cloth, cotton balls, cotton swabs, and/or the like.

The kit 200 may be available to clinicians (or medical professionals) for use in a sealed and sterilized package. In some cases, the clinician may further apply a window dressing over the injection site 16 to provide additional protection from infection at the injection site 16. Further, the securement device 100 may allow the clinician to access the insertion site 16 by pivoting the base 102 relative to each of the pair of anchor assemblies 130 in the secured state A1 of each of the pair of anchor assemblies 130 without dislodging the catheter 10 from the skin 14 of the patient.

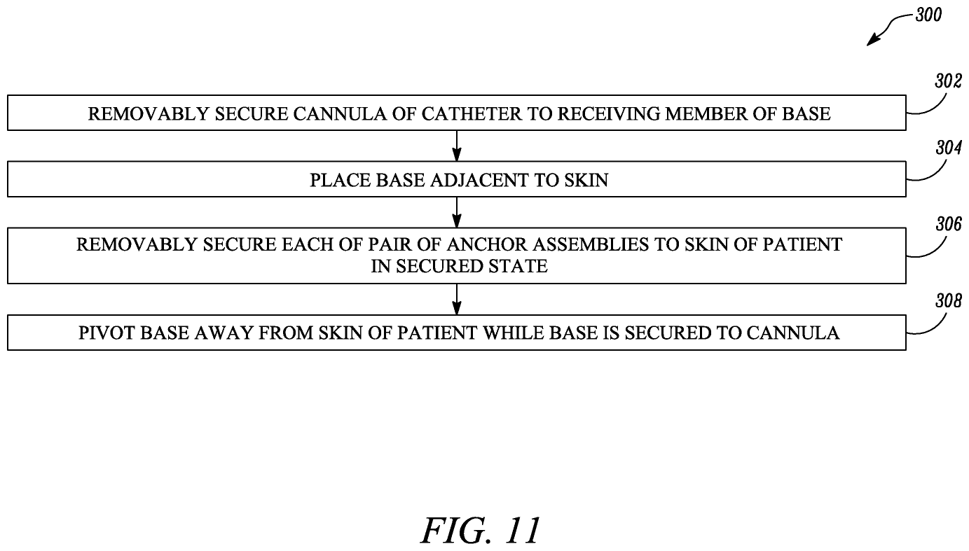

FIG. 11 is a flowchart illustrating a method 300 of using the securement device 100 with the catheter 10 having the cannula 12 inserted into the skin 14 of the patient at the insertion site 16. The method 300 will be described with reference to the catheter 10 of FIG. 1 and the securement device 100 of FIGS. 2-10.

Referring now to FIGS. 1-11, in some examples, the securement device 100 includes the base 102 having the receiving member 104, the pair arms 120 extending from the receiving member 104, and the pair of anchor assemblies 130 corresponding to the pair of arms 120. In some examples, each of the pair of anchor assemblies 130 is pivotally coupled to the corresponding arm 120 from the pair of arms 120 of the base 102.

At step 302, the method 300 includes removably securing the cannula 12 of the catheter 10 to the receiving member 104 of the base 102. In some examples, removably securing the cannula 12 of the catheter 10 to the receiving member 104 includes inserting the cannula 12 in the first slot 106 of the receiving member 104. In some examples, removably securing the cannula 12 of the catheter 10 to the receiving member 104 further includes removably coupling the cap 110 to the receiving member 104, such that the first slot 106 of the receiving member 104 and the second slot 112 of the cap 110 collectively and at least partially receive the cannula 12 therebetween.

At step 304, the method 300 further includes placing the base 102 adjacent to the skin 14. At step 306, the method 300 further includes removably securing each of the pair of anchor assemblies 130 to the skin 14 of the patient in the secured state A1. In some examples, removably securing each of the pair of anchor assemblies 130 to the skin 14 of the patient includes rotating the anchor ring 150 of each of the pair of anchor assemblies 130 along the first rotational direction R1, such that the arcuate penetrating portion 152 of the anchor ring 150 at least partially and removably penetrates the skin 14 of the patient. In some examples, removably securing each of the pair of anchor assemblies 130 to the skin 14 of the patient includes aligning the common pivot axis Y-Y′ of the pair of arms 120 with the insertion site 16, such that the insertion site 16 is disposed between the pair of arms 120.

At step 308, the method 300 further includes pivoting the base 102 away from the skin 14 of the patient while the base 102 is secured to the cannula 12. In some examples, the method 300 further includes performing a maintenance procedure at the insertion site 16 after pivoting the base 102 away from the skin 14 of the patient. In some examples, the maintenance procedure may include cleaning the skin 14 of the patient disposed adjacent to the insertion site 16. In some examples, the method 300 further includes pivoting each of the pair of arms 120 towards the skin 14 of the patient after performing the maintenance procedure, such that the base 102 is disposed adjacent to the skin 14.

In some examples, to remove the securement device 100, the clinician may rotate the anchor rings 150 along the second rotational direction R2 from the secured state A1 of each of the pair of anchor assemblies 130 to the released state A2. Subsequently, the clinician may uncouple the cap 110 from the receiving member 104 to release the cannula 12 of the catheter 10 from the base 102.

The securement device 100 of the present disclosure may secure and stabilize the catheter 10 firmly onto the skin 14 of the patient. Further, the securement device 100 may restrict a movement of the cannula 12 with respect to the skin 14 of the patient during normal usage. Specifically, each of the pair of anchor assemblies 130 may prevent dislodgement of the catheter 10 or the cannula 12 when removably secured to the skin 14 of the patient through the arcuate penetrating portion 152 of the anchor ring 150. Moreover, each of the pair of anchor assemblies 130 may allow ease of removal of the securement device 100 from the the skin 14 of the patient by rotating the anchor rings 150 along the second rotational direction R2 and uncoupling the cap 110 from the receiving member 104.

In the secured state A1 of each of the pair of anchor assemblies 130, the securement device 100 may allow the base 102 to be pivoted relative to the pair of anchor assemblies 130 while being secured to the cannula 12, thereby allowing all around access to the insertion site 16 for cleaning and maintenance purposes without the need to dislodge the catheter 10 from the securement device 100. Further, the securement device 100 may be secured to the skin 14 of the patient away from the insertion site 16 allowing minimal interference with the insertion site 16. The securement device 100 may be used for extended period of time without causing discomfort to the patient, thereby enhancing a user experience.

Additionally, the securement device 100 may allow a length (e.g., the exposed length 178) of the cannula 12 between the base 102 and the insertion site 16 to be fixed, thereby eliminating pistoning of the cannula 12 during pivoting movement of the base 102 relative to the pair of anchor assemblies 130. Further, the securement device 100 of the present disclosure may eliminate the need for tapes, patches, and/or sutures to secure the catheter 10 onto the skin 14 of the patient, thereby preventing medical adhesive-related skin injury (MARSI) complications. Further, the base 102 of the securement device 100 may removably secure the cannula 12 of various sizes and geometries, thereby enabling compatibility with multiple brands and applications.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below, or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example.

Various examples have been described. These and other examples are within the scope of the following claims.

1. A securement device for a catheter having a cannula configured to be inserted into a skin of a patient at an insertion site, the securement device comprising:
a base comprising:
a receiving member configured to be removably secured to the cannula; and
a pair of arms extending from the receiving member and opposing each other; and
a pair of anchor assemblies corresponding to the pair of arms and spaced apart from each other, wherein each of the pair of anchor assemblies is pivotally coupled to a corresponding arm from the pair of arms of the base, and wherein each of the pair of anchor assemblies is configured to be removably secured to the skin of the patient in a secured state;
wherein, in the secured state of each of the pair of anchor assemblies, the base is pivotable relative to the pair of anchor assemblies while being secured to the cannula.

2. The securement device of claim 1, wherein the receiving member comprises a first slot configured to at least partially and removably receive the cannula therein.

3. The securement device of claim 2, further comprising a cap configured to be detachably coupled to the receiving member and comprising a second slot, wherein, upon detachable coupling of the cap to the receiving member, the second slot is at least partially aligned with the first slot, and wherein, upon insertion of the cannula in the first slot and detachable coupling of the cap to the receiving member, the first and second slots collectively and at least partially receive the cannula therebetween, such that the cannula is removably secured to the receiving member.

4. The securement device of claim 3, further comprising a first sleeve at least partially received within the first slot and a second sleeve at least partially received within the second slot, wherein, upon insertion of the cannula in the first slot and detachable coupling of the cap to the receiving member, each of the first sleeve and the second sleeve frictionally engages with the cannula, such that the cannula is removably secured to the receiving member.

5. The securement device of claim 3, wherein the cap is detachably coupled to the receiving member by a snap-fit connection, and wherein the cap comprises a lip configured to be manually gripped for releasing the cap from the receiving member.

6. The securement device of claim 3, wherein the base further comprises a living hinge pivotally coupling the cap to the receiving member.

7. The securement device of claim 2, wherein the receiving member further comprises a longitudinal axis extending along its length, and wherein the first slot at least partially extends along the longitudinal axis, such that the cannula at least partially extends from the receiving member along the longitudinal axis.

8. The securement device of claim 7, wherein the longitudinal axis corresponds to an axis of symmetry of the base, such that the pair of arms are symmetrically arranged about the longitudinal axis.

9. The securement device of claim 7, wherein each of the pair of arms curves away from the longitudinal axis, such that the cannula is at least partially disposed between the pair of arms.

10. The securement device of claim 7, wherein each of the pair of arms is pivotable about a common pivot axis that is substantially orthogonal to the longitudinal axis, and wherein the longitudinal axis and the common pivot axis intersect each other at or directly above the insertion site.

11. The securement device of claim 10, wherein each of the pair of anchor assemblies comprises a planar support surface configured to be disposed on the skin of the patient and defining a support plane parallel to the common pivot axis, wherein, in a home state of the base, the longitudinal axis is parallel to the support plane while being secured to the cannula, and wherein, in a maintenance state of the base, the longitudinal axis is inclined to the support plane while being secured to the cannula.

12. The securement device of claim 11, wherein each of the pair of arms comprises an alignment line orthogonal to the longitudinal axis, and wherein, in the home state of the base, the alignment line is further orthogonal to the common pivot axis.

13. The securement device of claim 1, wherein each of the pair of anchor assemblies comprises:
an anchor wheel pivotally coupled to the corresponding arm;
an anchor guide fixedly coupled to and disposed around the anchor wheel, wherein the anchor guide comprises an arcuate slot extending therethrough; and
an anchor ring comprising an arcuate penetrating portion slidably and at least partially received within the arcuate slot of the anchor guide, such that the anchor ring is rotatable relative to the anchor guide along the arcuate slot, wherein the arcuate penetrating portion comprises a sharp tip configured to removably penetrate the skin of the patient;
wherein, in the secured state of the corresponding anchor assembly, the arcuate penetrating portion at least partially penetrates the skin of the patient in order to removably secure the corresponding anchor assembly to the skin of the patient.

14. The securement device of claim 13, wherein the anchor ring is rotatable relative to the corresponding arm between the secured state and a released state of the corresponding anchor assembly, wherein, in the released state of the corresponding anchor assembly, the arcuate penetrating portion is removed from the skin of the patient, such that the corresponding anchor assembly is released from the skin of the patient, wherein the anchor ring is rotatable along a first rotational direction to switch the corresponding anchor assembly from the released state to the secured state, and wherein the anchor ring is rotatable along a second rotational direction opposite to the first rotational direction to switch the corresponding anchor assembly from the secured state to the released state.

15. The securement device of claim 14, wherein at least one of the anchor wheel and the anchor guide comprises one or more indicia indicative of the first rotational direction, the second rotational direction, the secured state, and the released state.

16. The securement device of claim 13, wherein the anchor ring of each of the pair of anchor assemblies is rotatable about a common rotation axis, and wherein each of the pair of arms is pivotable about a common pivot axis coaxial with the common rotation axis.

17. The securement device of claim 13, wherein the anchor guide of each of the pair of anchor assemblies comprises an alignment mark aligned with the insertion site.

18. The securement device of claim 13, wherein the anchor wheel comprises one or more first locking features, wherein the anchor ring comprises one or more second locking features complimentary to the one or more first locking features, and wherein, in the secured state of the corresponding anchor assembly, the one or more first locking features are configured to releasably and lockingly engage with the one or more second locking features to prevent the anchor ring from rotating relative to the corresponding arm.

19. The securement device of claim 13, wherein the anchor wheel comprises an arcuate shoulder, wherein the anchor ring comprises a projection slidably engaging with the arcuate shoulder, and wherein, upon rotation of the anchor ring, the projection of the anchor ring slides along the arcuate shoulder.

20. The securement device of claim 13, wherein the anchor wheel of each of the pair of anchor assemblies comprises a protrusion, wherein each arm comprises a collar proximal to an end distal to the receiving member, and wherein the collar of each arm at least partially and rotatably receives the protrusion of the anchor wheel of the corresponding anchor assembly to pivotally couple each arm to the corresponding anchor assembly.

21. The securement device of claim 20, wherein the anchor ring further comprises:
a coupling portion defining an aperture therethrough and disposed adjacent to the anchor wheel, wherein the aperture of the coupling portion at least partially receives the protrusion of the anchor wheel therethrough to rotatably couple the anchor ring to the anchor wheel;
an intermediate portion extending radially from the coupling portion, wherein the arcuate penetrating portion extends from the intermediate portion radially distal to the coupling portion; and
a grip pad coupled to and at least partially disposed around the intermediate portion, wherein the grip pad comprises a plurality of ridges configured to be manually gripped for rotating the anchor ring relative to the anchor guide.

22. The securement device of claim 21, wherein the coupling portion is disc-shaped.

23. The securement device of claim 21, wherein the intermediate portion tapers in a radially inward direction from the grip pad to the coupling portion.

24. The securement device of claim 1, wherein each of the pair of arms is rotatable in an angular range of about 180 degrees relative to the corresponding anchor assembly.

25-54. (canceled)
